(19) United States
(12) Patent Application Publication
(10) Pub. No.: US 2002/0137124 A1
(43) Pub. Date: Sep. 26, 2002

(54) Process for the continuous production of dipeptide crystals in a membrane and hydro-cyclone reactor using a peptidase

(76) Inventors: Duarte Miguel Franca Teixeira Dos Prazeres, Lisboa (PT); Ana Isabel Jorge Dias, Lisboa (PT); Ana Sofia Feliciano Santos, Lisboa (PT); Joaquim Manuel Sampaio Cabral, Lisboa (PT); Maria Luisa Mourato Serraiheiro, Lisboa (PT)

Correspondence Address: SUGHRUE MION, PLLC, 2100 PENNSYLVANIA AVENUE, N.W., WASHINGTON, DC, 20037, US

(21) Appl. No.: 10/031,168

(22) PCT Filed: May 17, 2001
(86) PCT No.: PCT/PT01/00008

(30) Foreign Application Priority Data
May 17, 2000 (PT) 102469

(51) Int. Cl. C12P 21/06; C07K 5/06

(52) U.S. Cl. 435/68.1; 562/455; 562/561

(57) ABSTRACT

The invention relates to a process for the production of dipeptide crystals (generic formula AcXYNH₂) with high purity (>95 %), by enzymatic synthesis with a protease in organic media of reversed micelles, starting from derivatives of the two constituent amino acids (AcXOEt and YNH₂). A membrane and hydro-cyclone reactor was designed which enables the continuous and simultaneous synthesis and crystallisation of the dipeptides. The dipeptide crystals thus prepared are continuously removed and further separated from the remaining liquid by filtration or centrifugation. After drying, the crystals are dissolved in hot methanol. By decreasing the temperature, the dipeptide re-crystallises, originating a high purity product. After re-crystallisation the product is filtered and dried.

### FIELD OF THE INVENTION

The invention relates to a process for the production of dipeptide (with generic formula AcXYNH₂) crystals with high purity (>95%), by enzymatic synthesis with a protease in organic media of reversed micelles, starting from derivatives (AcXOEt and YNH₂) of the two constituting amino acids (X and Y). An enzymatic, membrane and hydro-cyclone, reactor was designed which is suitable for the reaction system, and which enables the continuous and simultaneous synthesis and crystallisation of the dipeptides. This invention belongs to the technical domain of Biochemical Engineering/Biotechnology.

### BACKGROUND TO THE INVENTION

Numerous peptides—short chains with 2 to 50 amino acids—have been identified in the last 20 years, which are biologically active and play important roles in the control and regulation of biological processes. These peptides can act as neurotransmitters, hormones, antibiotics and immunologic or anti-cancerigenous agents. Several medicinal products already exist which have a peptidic nature and the perspectives for the development and discovery of others are enormous. There is nowadays a market in the research area with a growing appetite for the purchase of biologically active peptides. Although some of the peptides occur naturally, the vast majority is synthesised chemically by coupling the constituent amino acids (a.a.). Two approaches are usually used to perform peptide synthesis, the chemical synthesis in solution and the chemical synthesis in solid state of Merrifield. One of the major disadvantages of chemical synthesis lies in its inherent low selectivity and in the need to subsequently perform costly purification steps (mostly high-pressure liquid chromatography—HPLC) which represent an important fraction of the total product cost (more than 50%).

### DESCRIPTION OF THE INVENTION

The production technology described here presents several innovative aspects, which when taken together enable the production of dipeptides (chains with 2 a. a.) with a high purity. Firstly, the process takes advantage of the higher selectivity of biological catalysts that originate a lower amount of secondary products. Apart from this advantage, and particularly important, is the fact that the first step of production lies in a innovative concept of integrated process, which explores the possibility of performing simultaneously and in the same unit, the synthesis and purification steps. This way it is possible to reduce drastically the purification costs, and thus the final product cost. Another advantage of the process lies in its simplicity and in the low cost of the production equipment needed.

The process refers to the synthesis of dipeptides starting from two generic amino acid (X and Y) derivatives, of the type AcXOEt (the ethylic ester of the acetylated amino acid X) and YNH₂ (the amide of amino acid Y). The designation Ac refers to the acetyl group and the designation OEt to the ethylic ester group. The synthesis is catalysed by a protease or another enzyme with the ability to synthesise peptide bonds. Proteases such as α-chymotrypsin, trypsin or papain can be used. The synthesis may also be accomplished chemically, without the use of enzymes.

The enzyme and the amino acid with hydrophilic character (YNH₂) are dissolved in an aqueous buffer. A certain volume of this solution is added to a mixture of organic solvents (alcane plus alcohol) containing a surfactant. Alcanes such as heptane, isooctane or octane, alcohols such as hexanol, octanol or dodecanol and surfactants such as dodecyl-trimethyl ammonium bromide, tetradecyl-trimethyl ammonium bromide or hexadecyl-trimethyl ammonium bromide can be used. This mixture is stirred until the complete solubilisation of the aqueous solution in the organic solvent, under the form of microscopic spherical structures termed reversed micelles.

The second amino acid, with a hydrophobic character (AcXOEt) is dissolved in the same mixture of organic solvents (alcane plus alcohol). The synthesis reaction starts when the solutions containing the two amino acids are added in a reactor (in batch or continuous mode). The same reaction also occurs in the absence of the enzyme, but at a much lower rate. Two secondary products are formed together with the dipeptide (see scheme): a) a product (AcXOH) which results from the hydrolysis of the amino acid derivative AcXOEt, b) a product (AcXOR) which results from the transesterification reaction of the same amino acid derivative (AcXOEt) with one of the solvents (the alcohol ROH).

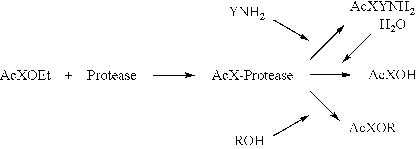

The composition of the media of reversed micelles in organic solvents (type and concentration of surfactant, alcane, alcohol and buffer; concentration of water and enzyme), is controlled in such a way that: a) the side reactions which originate the secondary products are minimised, b) the rate and yield of synthesis of dipeptide AcXYNH₂ are maximised, c) the solubility of the produced dipeptides is minimised. This composition may vary as a function of the specific dipeptide that is being produced. Examples of the products covered by the invention are:

N-acetyl-L-phenylalanine leucinamide (AcPheLeuNH₂), N-acetyl-L-phenylalanine isoleucinamide (AcPheIleNH₂), N-acetyl-L-phenylalanine valinamide (AcPheValNH₂), N-acetyl-L-phenylalanine alaninamide (AcPheAlaNH₂), N-acetyl-L-phenylalanine phenylalaninamide (AcPhePheNH₂), N-acetyl-L-phenylalanine methioninamide (AcPheMetNH₂), N-acetyl-L-tyrosine leucinamide (AcTyrLeuNH₂), N-acetyl-L-tyrosine isoleucinamide (AcTyrIleNH₂), N-acetyl-L-tyrosine valinamide (AcTyrValNH₂), N-acetyl-L-tyrosine methioninamide (AcTyrMetNH₂), N-acetyl-L-tryptofan leucinamide (AcTrpLeuNH₂), N-acetil-L-tryptofan isoleucinamide (AcTrpIleNH₂), N-acetyl-L-tryptofan valinamide (AcTrpValNH₂).

This type of system/reaction has been described in the literature (Serralheiro and Cabral, 1992; 1994; Serralheiro et al., 1994; 1999; Feliciano et al., 1997a; 1997b).

The invention refers to the process and reactor used to carry out the synthesis of dipeptides of the type AcXYNH₂. FIG. 1 present a schematic drawing of the proposed reactor. The reactor consists of a module (D, FIG. 1) containing a tubular ultrafiltration membrane, coupled to a glass vessel with a cylindrical geometry and a conic bottom named hydro-cyclone (B, FIG. 1).

The ultrafiltration membrane (2) is made of a ceramic material (e.g. Carbosep®) or other material characterised by the fact that it is resistant to organic solvents. The solutions containing each of the amino acids are added to the system. The temperature is kept constant at a value that is adequate for the enzymatic activity and stability, and for the crystallisation. The mixture is removed from the centre of the vessel by a central tube (4), pumped (C2) through the cylindrical membrane (2) and back to the cylindrical vessel. By the action of the pressure, the liquid crosses the membrane pores and is collected in vessel (E). The enzyme is retained by the ultrafiltration membrane, selected in such a way that the diameter of the pores is inferior to the size of the enzyme.

The synthesis reaction occurs in the cylindrical vessel (B) and in the membrane module (D). When the concentration of the dissolved dipeptide reaches the supersaturation value, the formation of crystals starts. Seed crystals may also be added as a way of starting the crystallisation. The dipeptide crystals do not cross the membrane due to their size. The secondary products AcXOH and AcXOR remain in solution, exiting the reactor by permeation across the membrane and being collected in a vessel (E). A fraction of reagents AcXOEt and YNH₂, also exits the system by permeation across the membrane. The reaction mixture enters the hydro-cyclone through a side entrance (entrance 3 in FIG. 1) which is put tangentially to the wall of the hydro-cyclone. In this way the mixture acquires a centrifugal motion which pushes the crystal particles towards the walls of the vessel. Upon contact with the walls the crystals loose momentum and tend to sediment. For this reason, the mixture in the top of the vessel remains partially clarified while the crystals accumulate in the bottom of the vessel, from where they can be removed during the process through a pump (C3), being collected in a vessel (F). The dimensions of the hydro-cyclone are chosen according to the usual design parameters and in such a way that the sedimentation process is the most efficient possible.

A mixture of the two amino acids in reversed micellar media is added continuously to the hydro-cyclone by a pump (C1) from vessel (A). The rate of addition of this media is equal to the permeation rate of the liquid across the membrane, and thus the liquid level in the reactor is maintained constant.

The proposed process/reactor enables the integration of the dipeptide enzymatic synthesis and purification steps in a single operation. The composition of the reversed micellar media in organic solvents (type and concentration of surfactant, alcane and alcohol) is controlled in such a way that the synthesised dipeptides have a very low solubility in the media. This composition may vary as a function of the dipeptide to be produced. The low solubility enables the occurrence of crystallisation simultaneously with the synthesis. The crystallisation is advantageous because: a) it removes the dipeptide from the liquid phase thus reducing its degradation by secondary hydrolysis, b) it purifies the product significantly, thus reducing the number of purification operations subsequently needed to achieve the specified degree of purity.

The reactor design explores the principle of operation of hydro-cyclones, enabling the continuous sedimentation of a fraction of the crystals in the conic bottom of the reactor. The crystals may thus be continuously removed during the production process, without affecting the synthesis reaction and without the need to suspend the operation. The reactor was designed in order to maximise the sedimentation in the bottom of the reactor and the clarification in the top. On the other hand, the presence of an ultrafiltration membrane makes it possible to retain the enzyme in the system and thus operate the process continuously.

Characteristics which are considered innovative-the process developed possesses some characteristics which are considered innovative, namely: a) the combination of a synthesis reaction with the simultaneous crystallisation of the product formed; b) the coupling of an ultrafiltration module with a hydro-cyclone, that enables the existence of two outlet streams from the reactor, one for the removal of the secondary products and the other for the removal of the product in its crystalline form.

### EXAMPLE 1

Nine ml of a reversed micellar solution were prepared with the following composition: 89.1% (volume) heptane, 9.9% (volume) octanol, 1.0% (volume) carbonate buffer (20 mM, pH 10), 0.1M tetradecyl trimethyl ammonium bromide, 0.55 μM α-chymotrypsin and 10 mM LeuNH₂ (leucinamide). The solution was placed in a cylindrical vessel with the temperature controlled at 15° C. and an agitation speed of 400 rpm. The reaction was started by the addition of 1 ml of a reversed micellar solution with the following composition: 89.1% (volume) heptane, 9.9% (volume) octanol, 1.0% (volume) carbonate buffer (20 mM, pH 10), 0.1M tetradecyl trimethyl ammonium bromide and 30 mM AcPheOEt (ethylic ester of N-acetyl-L-phenylalanine). The final composition of the system was: 89.1% (volume) heptane, 9.9% (volume) octanol, 1.0% (volume) carbonate buffer (20 mM, pH 10), 0.1M tetradecyl trimethyl ammonium bromide, 0.50μM α-chymotrypsin, 9 mM LeuNH₂ and 3 mM AcPheOEt. The reaction was carried out during one hour until the complete consumption of the amino acid AcPheOEt. The process was monitored by HPLC. At the end of the reaction the crystals formed were recovered by centrifugation at 4000 rpm. The reaction yield in the dipeptide AcPheLeuNH₂ was 85%. Around 89% of this product was recovered in the form of crystals with a purity higher than 98%.

### Example 2

The hydro-cyclone (B, FIG. 1) was loaded with 270 ml of a reversed micellar solution with the following composition: 89.1% (volume) heptane, 9.9% (volume) octanol, 1.0% (volume) carbonate buffer (20 mM, pH 10), 0.1 M tetradecyl trimethyl ammonium bromide, 0.55 μM α-chymotrypsin and 10 mM IleNH₂ (isoleucinamide). The mixture was re-circulated through the system with pump C2 at a flow rate of 500 ml/min during 20 minutes. The reaction was initiated by the addition of 30 ml of a reversed micelllar solution with the following composition: 89.1% (volume) heptane, 9.9% (volume) octanol, 1.0% (volume) carbonate buffer (20 mM, pH 10), 0.1M tetradecyl trimethyl ammonium bromide and 30 mM AcPheOEt (ethylic ester of N-acetyl-L-phenylalanine). The final composition of the system was: 89.1% (volume) heptane, 9.9% (volume) octanol, 1.0% (volume) carbonate buffer (20 mM, pH 10), 0.1 M tetradecyl trimethyl ammonium bromide, 0.50μM α-chymotrypsin, 9 mM IleNH₂ and 3 mM AcPheOEt. The system was operated in a discontinuous mode (pump C1 off and re-circulation of the permeate stream back to the hydro-cyclone) at 15° C. during 1 hour. The continuous operation was then initiated by introducing the following mixture of reagents from vessel A through pump C1: 89.1% (volume) heptane, 9.9 % (volume) octanol, 1.0 % (volume) carbonate buffer (20 mM, pH 10), 0.1M tetradecyl trimethyl ammonium bromide, 9 mM IleNH₂ and 3 mM AcPheOEt. The feed flow rate was approximately 0.25 ml/min, identical to the flow rate of permeate which was now recovered in vessel E. During the operation crystals of the dipeptide AcPheIleNH₂ were being formed and accumulating in the bottom of the hydro-cyclone. Volumes of 10 ml of the mixture containing the crystals were periodically removed from the bottom of the hydro-cyclone (pump C3). The crystals in this mixture were separated by centrifugation and the supernatant was returned to the hydro-cyclone. In this operation a total of 3.03 grams of AcPheIleNH₂ were produced. The final volume of the mixture was recovered and centrifuged and a mass of 3.8 grams of crystals was obtained. After re-crystallisation from hot methanol around 1.27 grams of AcPheIleNH₂ were obtained with a purity >99% (measured by high-pressure liquid chromatography'HPLC).

### REFERENCES

Jones, J. 1992, Amino acid and peptide synthesis. In: Oxford Chemistry Primers Series, N^{o}7, S. G. Davies (ed.), Oxford Science Publishers, Oxford.

Feliciano, A. S., Cabral, J. M. S. and Prazeres, D. M. F. Quantitative structure activity relationships in the synthesis of AcXYNH₂ dipeptides by α-chymotrypsin in reversed micelles. Enzyme Microb. Technol. 1997a, 21, 284-290

Serralheiro, M. L. M. and Cabral, J. M. S. Application of fractional factorial design to the study of enzymatic dipeptide synthesis in reverse micelles. In: Biocatalysis in Non-Conventional Media (Tramper, J., Vermue, M. H., Beeftink, H. H. and Stockar, U., eds.). Elsevier, Amsterdam, 1992, 725-732

Serralheiro, M. L. M. and Cabral J. M. S. Synthesis of AcPheLeuNH₂ by α-chymotrypsin in TTAB reversed micelles: application of response surface methodology to the optimization of the system. Biotechnol. Bioeng. 1994, 43, 103 1-1042

Serralheiro, M. L. M., Prazeres, D. M. F. and Cabral, J. M. S. Dipeptide synthesis and separation in a reversed micellar membrane reactor. Enzyme Microb. Technol. 1994, 16,1064-1073

Serralheiro, M. L. M., Prazeres, D. M. F. and Cabral, J. M. S. Continuous Production and simultaneous precipitation of a dipeptide in a reversed micellar membrane reactor, Enzyme Microb. Technol., 1999, 24, 507-413.

Feliciano, A. S., Cabral, J. M. S. and Prazeres, D. M. F. Solubility studies and synthesis of AcPheLeuNH₂ in reversed micellar systems. Biocatal. Biotrans. 1997b, 14, 219-234

### DESCRIPTION OF THE FIGURE

FIG. 1: Schematic representation of the enzymatic membrane and hydro-cyclone reactor for the simultaneous synthesis and crystallisation of dipeptides in reversed micellar media A: reagent reservoir, B: hydro-cyclone made of glass or other material resistant to solvents, C1, C2 e C3: pumps, D: cylindrical module containing the tubular ceramic membrane, E: reservoir with by-products and unconverted reagents, F: reservoir with dipeptide crystals, 1: cylindrical ultrafiltration module, 2: tubular ceramic membrane, 3: tangential side entrance, 4: central outlet tube.

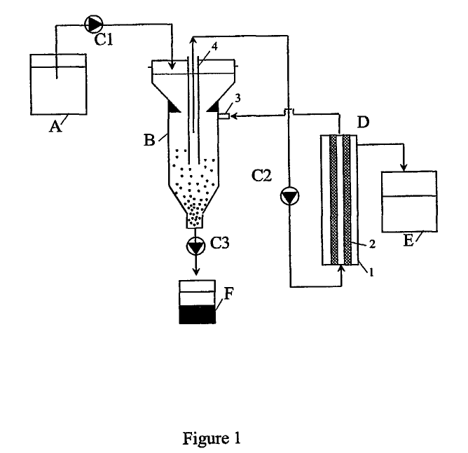

1. An integrated process of simultaneous synthesis and crystallisation for the preparation of dipeptide crystals with the generic formula AcXYNH₂, starting from the amino acids AeXOEt and YNH₂, which is characterised by the use of proteases in organic media of reversed micelles.

2. Process according to 1, characterised by the fact that it is carried out in batch reactors.

3. Process according to 1, characterised by the fact that it is carried out in an enzymatic, membrane and hydro-cyclone, reactor which enables the continuous or batch production of the referred crystals.

4. Process according to claims 1, 2 and 3 characterised by the fact that it is used for the production of other dipeptides or dipeptide derivatives with the generic formula XY, starting from amino acids X and Y.

5. Process according to claims 1, 2, 3 and 4 characterised by the fact that the synthesis process is chemical and not enzymatic.

6. Process according to claims 3, 4 or 5, characterised by the fact that instead of a hydro-cyclone, another unit is coupled to the system which also enables the continuous sedimentation and removal of the crystals from the system.

7. Process according to claims 1, 2, 3, 4, 5 and 6 characterised by the fact that the resulting crystals are further purified by re-crystallisation in an adequate solvent.
